(19) United States
(12) Patent Application Publication
(10) Pub. No.: US 2025/0064476 A1
(43) Pub. Date: Feb. 27, 2025

(54) FLUID-COOLED ULTRASONIC SURGICAL INSTRUMENTS

(71) Applicant: Covidien LP, Mansfield, MA (US)

(72) Inventor: Matthew S. COWLEY, Boulder, CO (US)

(73) Assignee: Covidien LP, Mansfield, MA (US)

(21) Appl. No.: 18/729,428

(22) PCT Filed: Jan. 20, 2023
(86) PCT No.: PCT/IB2023/050492

§ 371 Date: Jul. 16, 2024

Related U.S. Application Data
(60) Provisional application No. 63/302,525, filed on Jan. 24, 2022.

(51) Int. Cl. A61B 17/32

(52) U.S. Cl. CPC A61B 17/320068; A61B 17/320016

(57) ABSTRACT

An ultrasonic surgical system includes a waveguide defining first and second longitudinal lumens extending through at least a portion of a length of the waveguide. Inflow and outflow conduits are fluidly coupled with the first and second longitudinal lumens to enable the inflow of fluid into the first longitudinal lumen and the outflow of fluid from the second longitudinal lumen. A coupler forms a fluid-tight seal between the inflow conduit and the first longitudinal lumen and between the outflow conduit and the second longitudinal lumen.

### FIELD

The present disclosure relates to ultrasonic surgical instruments and, more particularly, to devices, systems, and methods of manufacturing fluid-cooled ultrasonic surgical instruments.

### BACKGROUND

Ultrasonic surgical instruments and systems utilize ultrasonic energy, i.e., mechanical vibration energy transmitted at ultrasonic frequencies, to treat, e.g., seal and/or transect, tissue. Ultrasonic surgical instruments typically include a waveguide having a transducer coupled to a proximal end portion of the waveguide and an end effector disposed at a distal end portion of the waveguide. The waveguide transmits the ultrasonic energy produced by the transducer to the end effector for treating tissue at the end effector. The end effector may include a blade, hook, ball, etc. and/or other features such as a clamping mechanism for clamping tissue against the end effector and/or to facilitate manipulating tissue. During use, the waveguide and/or end effector of an ultrasonic surgical instrument can reach temperatures greater than 200° C. or even 300° C.

### SUMMARY

As used herein, the term “distal” refers to the portion that is described which is further from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is an ultrasonic surgical system including an ultrasonic waveguide, inflow and outflow conduits, and a coupler. The ultrasonic waveguide includes a distal body defining first and second longitudinal lumens in fluid communication with one another, and first and second transverse bores in fluid communication with the respective first and second longitudinal lumens. The inflow and outflow conduits are fluidly coupled with the respective first and second transverse bores to enable an inflow of fluid into the first longitudinal lumen and an outflow of fluid from the second longitudinal lumen. The coupler is configured to couple the inflow and outflow conduits to the distal body and form a fluid tight seal between the inflow and outflow conduits and the respective first and second transverse bores.

In aspects, the coupler may be welded to the distal body.

In aspects, the coupler may include a first main body and a first flange protruding radially outward from the first main body and welded to the distal body. The first main body may define a first channel therethrough that fluidly couples the first transverse bore and the inflow conduit to one another.

In aspects, the distal body may have an annular portion, and the first flange may be curved such that the first flange is flush with the annular portion.

In aspects, the distal body may have a flattened portion, and the first flange may be planar such that the first flange is flush with the flattened portion.

In aspects, the coupler may include a first coupler having the first main body and the first flange, and a second coupler. The second coupler may include a second main body, and a second flange protruding radially outward from the second main body and welded to the distal body. The second main body may define a second channel therethrough that fluidly couples the second transverse bore and the outflow conduit to one another.

In aspects, the first channel may be linear or L-shaped and may have a distal end portion of the inflow conduit received therein, and the second channel may be the other of linear or L-shaped and may have a distal end portion of the outflow conduit received therein.

In aspects, the coupler may include a cylindrical extension extending from the first main body and received within the first transverse bore.

In aspects, the first main body may be a ring that extends around a circumference of the distal body. The first main body may define a second channel therethrough that fluidly couples the second transverse bore and the outflow conduit to one another.

In aspects, the coupler may include a second flange that protrudes radially outward from the first main body and is welded to the distal body. The first and second flanges may extend from the first main body in opposite directions from one another.

In aspects, the ultrasonic waveguide may include a proximal body configured to connect to an ultrasonic transducer. The distal body may have a blade through which the first and second longitudinal lumens extend.

In aspects, the ultrasonic surgical system may further include a cooling system, a housing at least partially supporting the cooling system, and an elongated assembly extending distally from the housing and including the ultrasonic waveguide. The cooling system may be configured to pump cooling fluid through the inflow conduit into the first longitudinal lumen and/or pump cooling fluid through the second longitudinal lumen into the outflow conduit.

In aspects, the coupler may include an elastomeric over-mold overmolded to a distal end portion of each of the inflow and outflow conduits and the distal body.

In aspects, the coupler may include a manifold and a pair of compressible seals. The manifold may surround the distal body and may define first and second channels. The compressible seals may form a fluid-tight seal between the first channel and the first transverse bore and the second channel and the second transverse bore.

In aspects, the manifold may define a pair of annular recesses positioned around a respective opening of the first and second channels. The seals may be O-rings seated in the corresponding annular recesses.

In aspects, the coupler may include a first housing half surrounding a first section of the distal body, and a second housing half surrounding a second section of the distal body and having a second flange defining a channel. The channel of the first flange may be configured to fluidly couple the first transverse bore and the inflow conduit to one another, and the channel of the second flange may be configured to fluidly couple the second transverse bore and the outflow conduit to one another.

In aspects, the coupler may include a first gasket and a second gasket. The first gasket may be compressed between an end portion of the first flange and an outer surface of the first section of the distal body to form a fluid-tight seal between the channel of the first flange and the first transverse bore. The second gasket may be compressed between an end portion of the second flange and an outer surface of the second section of the distal body to form a fluid-tight seal between the channel of the second flange and the second transverse bore.

In accordance with another aspect of the present disclosure, a method of manufacturing an ultrasonic surgical system is provided and includes forming first and second longitudinal lumens through at least a portion of a length of a distal body of an ultrasonic waveguide; forming first and second bores transversely through the distal body into fluid communication with the respective first and second longitudinal lumens; and forming a fluid-tight seal between a coupler and the distal body. The first and second longitudinal lumens are in fluid communication, and the coupler is configured to fluidly couple inflow and outflow conduits to the respective first and second bores.

In aspects, forming the fluid-tight seal may include welding a flange of the at least one coupler to the distal body.

In aspects, forming the fluid-tight seal may include positioning a compressible seal between the inflow conduit and the first bore.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a perspective view of an ultrasonic surgical instrument provided in accordance with the present disclosure;

FIG. 2 is a side, cut-away view of a proximal portion of the ultrasonic surgical instrument of FIG. 1;

FIG. 3 is a schematic illustration of a robotic surgical system provided in accordance with the present disclosure;

FIG. 4A is a longitudinal cross-sectional view of an aspect of a waveguide of the ultrasonic surgical instrument of FIG. 1 with two types of couplers for coupling to a fluid-cooling system;

FIG. 4B is a perspective view illustrating a first of the couplers shown in FIG. 4A attached to the waveguide;

FIG. 4C is a perspective view illustrating a second of the couplers shown in FIG. 4A attached to the waveguide;

FIG. 5A is a perspective view illustrating another aspect of a waveguide of the ultrasonic surgical instrument of FIG. 1 with another pair of couplers for coupling to the fluid-cooling system;

FIG. 5B is an enlarged perspective view illustrating one of the couplers of FIG. 5A;

FIG. 6 is a perspective view illustrating another aspect of a waveguide of the ultrasonic surgical instrument of FIG. 1 with another coupler for coupling to the fluid-cooling system;

FIG. 7 is a longitudinal cross-sectional view of yet another aspect of a waveguide of the ultrasonic surgical instrument of FIG. 1 with inflow and outflow conduits overmolded thereto;

FIG. 8A is a side, perspective view illustrating another aspect of a waveguide of the ultrasonic surgical instrument of FIG. 1 showing still another aspect of a coupler for coupling to the fluid-cooling system;

FIG. 8B is a transverse cross-sectional view of a manifold of the coupler of FIG. 8A;

FIG. 9 is a transverse cross-sectional view of a manifold of yet another coupler of the ultrasonic surgical instrument of FIG. 1;

FIG. 10A is a side, perspective view illustrating still another aspect of a waveguide of the ultrasonic surgical instrument of FIG. 1 with another type of coupler for coupling to the fluid-cooling system; and

FIG. 10B is a longitudinal cross-sectional view of the coupler and waveguide of FIG. 10A.

### DETAILED DESCRIPTION

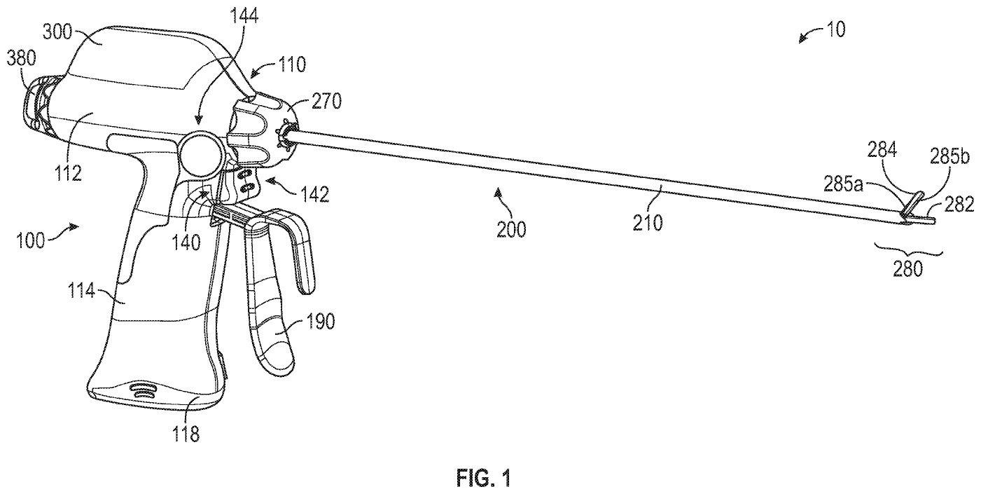

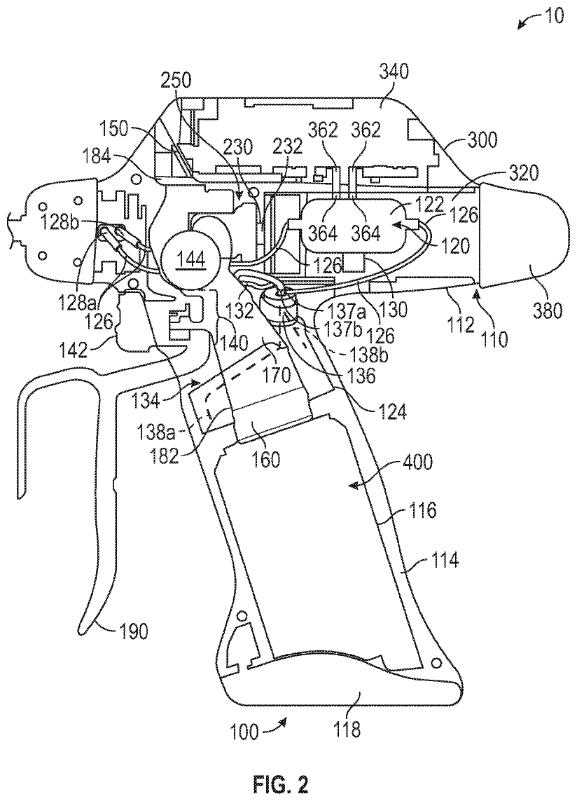

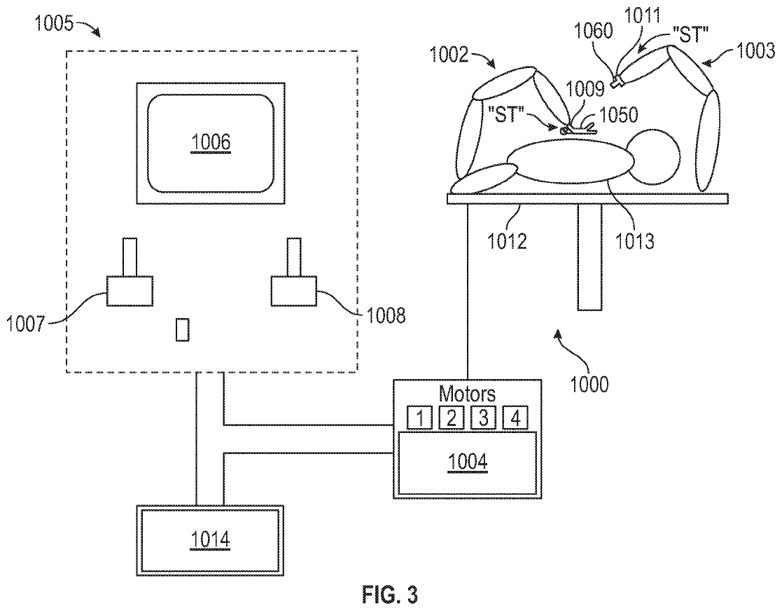

Turning to FIGS. 1 and 2, an ultrasonic surgical instrument provided in accordance with aspects of the present disclosure is generally identified by reference numeral 10. Instrument 10 is a fully cordless instrument that incorporates an on-board cooling system in addition to an on-board power source, e.g., battery, and an ultrasonic generator and transducer. However, it is also contemplated that instrument 10 be configured as a corded instrument, e.g., wherein instrument 10 is configured to connect to a remote cooling system by way of one or more fluid lines and to a remote ultrasonic generator (separate from or integrated with the remote cooling system) by way of a cable; or as a partially-corded instrument, e.g., wherein instrument 10 includes an on-board power source and ultrasonic generator and is configured to connect to a remote cooling system by way of one or more fluid lines, or wherein instrument 10 incorporates an on-board cooling system and is configured to connect to a remote ultrasonic generator. Likewise, other style ultrasonic instruments are also contemplated such as, for example, pencil-style instruments, hemostat-style instruments, etc. Thus, although particular aspects and features of instrument 10 are detailed below, it is understood that the aspects and features of the present disclosure are equally applicable for use with any other suitable ultrasonic surgical instrument or system, e.g., robotic surgical system 1000 (FIG. 3). Other suitable instruments for use in accordance with the present disclosure including remote or on-board cooling systems are described, for example, in Patent Application Pub. No. US 2019/0247073, titled “REMOVABLE FLUID RESERVOIR AND ULTRASONIC SURGICAL INSTRUMENT INCLUDING THE SAME” and filed on Feb. 13, 2018, and Patent Application Pub. No. US 2017/0281215, titled “DEVICES, SYSTEMS, AND METHODS FOR COOLING A SURGICAL INSTRUMENT” and filed on Mar. 18, 2017, the entire contents of each of which are hereby incorporated herein by reference.

Instrument 10 generally includes a handle assembly 100, an elongated assembly 200 that extends distally from handle assembly 100, a transducer and generator assembly (“TAG”) 300 configured for releasable engagement with handle assembly 100, and a battery 400 configured for removable receipt within handle assembly 100. Elongated assembly 200 may be integral with handle assembly 100 or may be releasably engagable with handle assembly 100.

Handle assembly 100 includes a housing 110, a cooling system 120, a switch assembly 140, a generator dock 150, a battery dock 160, a flex circuit assembly 170 (including flex circuit portions 182, 184), and a clamp trigger 190.

Housing 110 of handle assembly 100 includes a body portion 112 defining a longitudinal axis and a fixed handle portion 114 extending from body portion 112 at an oblique angle relative to the longitudinal axis of body portion 112 (although fixed handle portion 114 may alternatively extend perpendicularly relative to the longitudinal axis of body portion 112). Body portion 112 of housing 110 is configured to receive a proximal portion of elongated assembly 200 in operable engagement with clamp trigger 190 such that actuation of clamp trigger 190 manipulates end effector assembly 280 of elongated assembly 200. When engaged with body portion 112 of housing 110, elongated assembly 200 is aligned on the longitudinal axis of body portion 112. Body portion 112 of housing 110 is also configured to support TAG 300 thereon with transducer 320 of TAG 300 mechanically coupled with waveguide 230 of elongated assembly 200, e.g., via a threaded connection, latching, or in any other suitable manner, and both aligned on the longitudinal axis of body portion 112 of housing 110. Generator 340 of TAG 300 is electrically coupled with generator dock 150 of housing 110 when TAG 300 is engaged with body portion 112 of housing 110. Fixed handle portion 114 of housing 110 defines an internal compartment 116 configured to removably receive battery 400 therein and a hinged door 118 configured to enclose battery 400 within internal component 116.

Cooling system 120 includes one or more fluid pumps 122 and, in some aspects, a fluid reservoir 124, although fluid reservoir 124 may be omitted in other configurations. Cooling system 120 further includes associated tubing 126 operably interconnecting fluid pump 122, fluid reservoir 124, and inflow and outflow/return conduits 128a, 128b (or interconnecting fluid pump 122 and inflow and return conduits 128a, 128b, in aspects where fluid reservoir 124 is omitted) for pumping cooling fluid to and returning cooling fluid from elongated assembly 200. Inflow and return conduits 128a, 128b may extend along and/or through elongated assembly 200, ultimately entering waveguide 230 thereof to permit circulation of cooling fluid through blade 282 of end effector assembly 280 of elongated assembly 200.

The one or more fluid pumps 122 of cooling system 120 are supported within body portion 112 of housing 110. For example, a fluid pump 122 may be supported on either or both sides of body portion 112 of housing 110 at radially-spaced positions relative to the longitudinal axis of body portion 112 of housing 110. In such a configuration, fluid pumps 122 may define relatively thin, elongate configurations such that sufficient space is defined between the pumps 122 and/or the pump 122 and housing 110 to permit passage of transducer 320 of TAG 300 therebetween (in alignment on the longitudinal axis) while requiring minimal, if any, increase in the overall with dimension of body portion 112 of housing 110 to accommodate pump(s) 122.

A connection interface 130 of cooling system 120 for enabling power and/or control signals to be transmitted to pump(s) 122 is positioned so as not to interfere with TAG 300. Further, a connector 132, e.g., lead wire, cable, flex circuit, or other suitable connector, extends through body portion 112 of housing 110 to couple the connection interface 130 with flex circuit assembly 170 of handle assembly 100 to permit communication of power and control signals between pump(s) 122, generator 340, switch assembly 140, and/or battery 400. More specifically, pump(s) of cooling system 120 may be controlled via a controller of generator 340, battery 400, or a separate controller of cooling system 120, e.g., within control box 130. Regardless of the location and/or configuration, the controller is configured to control pump(s) 122 so as to maintain a flow of cooling fluid sufficient to cool blade 282 of end effector 280, to activate and deactivate cooling in response to manual inputs, and/or to implement automatic cooling (for example, upon deactivation of the supply of energy). The one or more fluid pumps 122 may be piezoelectric microfluidic pumps or other microfluidic pumps such as micro-peristaltic pumps, syringe pumps, etc. Regardless of the particular pump configuration utilized, the one or more fluid pumps 122 may, in aspects, be configured to generate sufficient flow rate of cooling fluid so as to cool waveguide 230 of elongated assembly 200 from an initial temperature of about 300° C. to about 100° C. (or about 120° C.) to a cooled temperature of about 70° C. to about 0° C. (or less than about 60° C.) in from about 0.5 seconds to about 2.5 seconds (or in less than about 2 seconds). However, other temperatures and/or cooling times are also contemplated.

Continuing with reference to FIGS. 1 and 2, fluid reservoir 124 is disposed within housing 110 of handle assembly 100. More specifically, fluid reservoir 124 is disposed within fixed handle portion 114 of housing 110 and is positioned between internal compartment 116 of fixed handle portion 114 and body portion 112 of housing 110. Fluid reservoir 124 may define a cut-out 134 within which at least a portion of clamp trigger 190 extends upon actuation thereof. As such, fluid reservoir 124 does not interfere with actuation of clamp trigger 190. It is also contemplated that fluid reservoir 124 be positioned in other locations, e.g., at a free end of fixed handle portion 114 such that battery 400 is disposed between fluid reservoir 124 and body portion 112.

Fluid reservoir 124 further includes a port 136 having an input 137a and an output 137b. Tubing 126 of cooling system 120 is coupled to input 137a and output 137b of fluid reservoir 124 to couple fluid reservoir 124 with fluid pump(s) 122 and inflow and return conduits 128a, 128b. More specifically, either or both of the ends 138a, 138b of the tubes of tubing 126 may extend through input 137a and output 137b and into fluid reservoir 124 such that the ends 138a, 138b of the tubes of tubing 126 are disposed at opposite sides, ends, or portions of fluid reservoir 124, thereby maximizing the spacing therebetween. This configuration inhibits the hotter, returned cooling fluid from being immediately pumped back out of fluid reservoir 124. Further, the ends 138a, 138b of the tubes of tubing 126 are positioned within fluid reservoir 124 relative to one another such that, regardless of the orientation of handle assembly 100, any air in fluid reservoir 124 is inhibited from entering inflow conduit 128a.

Switch assembly 140 of handle assembly 100 includes an energy activation button 142 operably positioned to electrically couple to flex circuit assembly 170. Flex circuit assembly 170 electrically couples switch assembly 140, battery 400, and TAG 300 with one another. Thus, when energy activation button 142 is activated in an appropriate manner, power is supplied from battery 400 to TAG 300. Energy activation button 142 may be configured for dual-mode activation such that, a first activation of energy activation button 142 drives TAG 300 in a “LOW” power mode, while a second, different activation of energy activation button 142 drives TAG 300 in a “HIGH” power mode. Other suitable activation configurations are also contemplated.

Switch assembly 140 of handle assembly 100 further includes, in aspects, a pair of cooling activation buttons 144 operably positioned on either side of housing 110. Flex circuit assembly 170 electrically couples connection interface 130 of cooling system 120 with switch assembly 140, battery 400, and TAG 300. Thus, activation of either or both of cooling activation buttons 144 initiates cooling. In aspects, multiple activations and/or particular activation patterns of cooling activation button(s) 144 may subsequently terminate cooling, switch between different cooling modes or programs, etc.

Generator dock 150 is disposed on body portion 112 of housing 110 and is positioned to electrically couple to generator 340 of TAG 300 upon engagement of TAG 300 with housing 110. Battery dock 160 is disposed within internal compartment 116 of fixed handle portion 114 of housing 110 and is positioned to electrically couple to battery 400 upon receipt of battery 400 within internal compartment 116. Docks 150, 160, flex circuit assembly 170 (including flex circuit portions 182, 184 thereof), and connector 132 of cooling system 120 electrically couple TAG 300, switch assembly 140, control box 130 of cooling system 120, and battery 400 with one another to enable communication of power and/or control signals therebetween. Flex circuit portion 182, more specifically, interconnects battery dock 160 with flex circuit assembly 170. The flexible configuration of flex circuit portion 182 enables routing of flex circuit 182 about fluid reservoir 124, which is disposed between flex circuit assembly 170 and battery dock 160. Flex circuit portion 184, on the other hand, electrically couples flex circuit assembly 170 with generator dock 150.

Referring still to FIGS. 1 and 2, clamp trigger 190 of handle assembly 100 of instrument 10 extends from body portion 112 of housing 110 in opposing relation relative to fixed handle portion 114 of housing 110. Clamp trigger 190 is pivotably coupled to body portion 112 of housing 110 and operably associated with elongated assembly 200 such that pivoting of clamp trigger 190 towards fixed handle portion 114 of housing 110 pivots jaw 284 of end effector assembly 280 of elongated assembly 200 from an open position to a clamping position for clamping tissue between jaw 284 and blade 282, which extends distally from waveguide 230 of elongated assembly 200.

Elongated assembly 200 generally includes a sleeve assembly having an outer sleeve 210 and an inner sleeve (not shown) disposed within outer sleeve 210, waveguide 230 extending through the inner sleeve (not shown), a drive assembly 250, a rotation assembly 270 operably disposed about outer sleeve 210, and end effector assembly 280 disposed at the distal end of the sleeve assembly. End effector assembly 280 includes, as noted above, blade 282 and jaw 284, which is operably coupled to outer sleeve 210 such that translation of outer sleeve 210 pivots jaw 284 relative to blade 282 between the open and clamping positions. Drive assembly 250 operably couples a proximal portion of outer sleeve 210 with clamp trigger 190 such that actuation of clamp trigger 190 pivots jaw 284 relative to blade 282 between the open and clamping positions. In the above-detailed configuration, the inner sleeve is a support sleeve and the outer sleeve is a drive sleeve, although the opposite configuration is also contemplated, as are other suitable drive mechanisms for pivoting jaw 284 relative to blade 282.

Jaw 284 of end effector assembly 280 includes a more-rigid structural body 285a and a more-compliant jaw liner 285b. Structural body 285a is pivotably coupled to the inner sleeve of elongated assembly 200 and operably coupled with outer sleeve 210 of elongated assembly 200 to enable the above-detailed translation of outer sleeve 210 to impart pivotal motion of jaw 284 relative to blade 282 to clamp tissue between jaw liner 285b of jaw 284 and blade 282. Jaw liner 285b is positioned to oppose blade 282 in the clamping position of jaw 284.

Waveguide 230 extends through the inner sleeve (not shown), includes blade 282 extending from the distal end thereof and includes a proximal end portion that is configured to operably couple to transducer 320, e.g., via a threaded connection, latching, or in any other suitable manner. Inflow and return conduits 128a, 128b, in aspects, may extend from housing 110 at least partially along and/or through elongated assembly 200 before fluidly coupling with an interior flow path defined within waveguide 230 or may fluidly couple with waveguide 230 within housing 110. Waveguide 230, the interior flow path defined within waveguide 230, and the connection of inflow and return conduits 128a, 128b to waveguide 230 are described in greater detail below.

TAG 300 and battery 400 are each removable from handle assembly 100 to facilitate disposal of handle assembly 100 or to enable sterilization of handle assembly 100. TAG 300 may be configured to withstand sterilization such that TAG 300 may be sterilized for repeated use. Battery 400, on the other hand, is configured to be aseptically transferred and retained within compartment 116 of fixed handle portion 114 of housing 110 of handle assembly 100 such that battery 400 may be repeatedly used without requiring sterilization thereof. Alternatively or additionally, battery 400 may be sterilized. In some configurations, TAG 300 (or a portion thereof, e.g., generator 340 or transducer 320) may be integral with housing 110.

TAG 300 includes ultrasonic transducer 320 and generator 340. A set of connectors 362 and corresponding rotational contacts 364 associated with generator 340 and ultrasonic transducer 320, respectively, enable data and drive signals to be communicated from generator 340 to transducer 320, e.g., the piezoelectric stack of transducer 320, to drive transducer 320. Battery 400 powers generator 340 to produce a drive signal, e.g., a high voltage AC signal, that is communicated to transducer 320. Transducer 320 converts the signal into mechanical motion that is output along waveguide 230 to blade 282 of end effector assembly 280. Transducer 320 further includes a rotation knob 380 disposed at a proximal end thereof to enable rotation of transducer 320 relative to generator 340 and handle assembly 100. Rotation knob 380 may also facilitate operable coupling of transducer 320 with elongated assembly 200.

With reference to FIG. 3, a robotic surgical system in accordance with the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST” supporting an end effector 1050, 1060. One of the surgical tools “ST” may be ultrasonic surgical instrument 10 (FIG. 1), wherein manual manipulation and actuation features are replaced with robotic inputs. In such configurations, robotic surgical system 1000 may include or be configured to connect to an ultrasonic generator, a power source, and cooling system. The other surgical tool “ST” may include any other suitable surgical instrument, e.g., an endoscopic camera, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

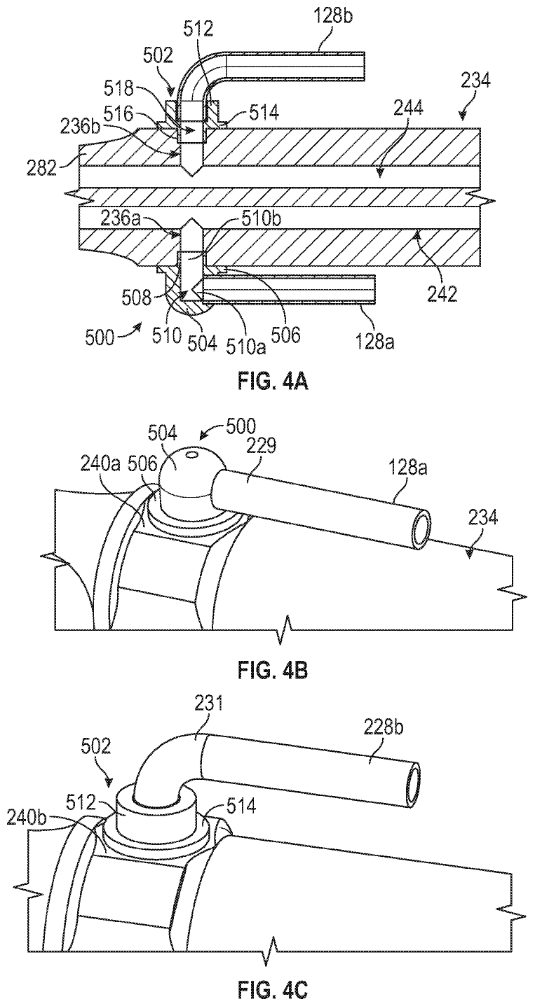

Referring to FIGS. 1, 2, and 4A-4C, waveguide 230, as noted above, includes blade 282 disposed at a distal end thereof. Waveguide 230, more specifically, includes a proximal body 232 (FIG. 2) and a distal body 234 (FIGS. 4A-4C) that includes blade 282. In aspects, proximal body 232 and distal body 234 may be integrally formed from a single piece of material or may be separately formed and subsequently attached to one another (permanently or removably). Proximal body 232 of waveguide 230 extends from housing 110 through at least a portion of the inner support sleeve of elongated assembly 200 to distal body 234. Proximal body 232 is configured to operable engage ultrasonic transducer 320 such that ultrasonic motion produced by ultrasonic transducer 320 is transmitted along proximal body 232 to distal body 234 and, ultimately, blade 282 for treating tissue clamped between blade 282 and jaw 284 or positioned adjacent to blade 282. Proximal body 232 may define a generally cylindrical-shaped configuration and may be solid, although hollow or semi-hollow configurations are also contemplated.

Distal body 234 may be formed from a solid material into which various lumens detailed below are formed, although other configurations are also contemplated. Distal body 234, more specifically, includes first and second longitudinal lumens 242, 244 extending in substantially parallel, spaced-apart relation relative to one another through at least a portion of blade 282. Longitudinal lumens 242, 244 merge with one another within a distal end of blade 282 to allow for incoming cooling fluid to pass into blade 282 via lumen 242 and outgoing cooling fluid to exit blade 282 via lumen 244. Lumens 242, 244 extend proximally from blade 282 to closed or open proximal ends. In aspects, a plug (not explicitly shown) may be positioned in each of lumens 242, 244 proximally adjacent respective transverse bores 236a, 236b (FIG. 4A) of distal body 234.

During manufacturing, in order to define a closed fluid circuit from cooling system 120, through waveguide 230, and back to cooling system 120 (see FIG. 2), inflow and return conduits 128a, 128b are in fluid communication with longitudinal lumens 242, 244, respectively using fluid-tight seals, such as, for example, first and second couplers 500, 502. Although the fluid circuit from cooling system 120 through waveguide 230 and back to cooling system 120 is closed, cooling system 120 may itself define an open-loop configuration (e.g., wherein supply fluid and return fluid are separate and return fluid is not re-circulated), a closed-loop configuration (e.g., wherein return fluid is re-circulated as supply fluid), or a semi-closed loop configuration (e.g., wherein some return fluid is re-circulated while other return fluid is not re-circulated).

With reference to FIGS. 4A and 4B, the first coupler 500 is secured to the distal body 234 of the waveguide 230 adjacent the first transverse bore 236a defined in the distal body 234, and the second coupler 502 is secured to the distal body 234 of the waveguide 230 adjacent the second transverse bore 236b defined in the distal body 234. The first and second transverse bores 236a, 236b are in fluid communication with the longitudinal lumens 242, 244, respectively. The distal body 234 of the waveguide 230 has a first flattened portion 240a (FIG. 4B) through which the first transverse bore 236a is formed, and a second flattened portion 240b (FIG. 4C) through which the second transverse bore 236b is formed.

The first coupler 500 includes a main body 504, an annular flange 506 protruding radially outward from the main body 502, and, in aspects, a cylindrical extension 508 extending into the first transverse bore 236a. In aspects, the main body 504, annular flange 506, and cylindrical extension 508 may be monolithically formed with one another or separate components that are connected to one another. The first coupler 500 defines an L-shaped channel 510 therethrough that fluidly connects the first transverse bore 236a and the inflow conduit 128a. More specifically, the L-shaped channel 510 has a first portion 510a extending substantially parallel with the lumen 242 and a second portion 510b that is substantially perpendicular to the first portion 510a and coaxial with the first transverse bore 236a. The first coupler 500 is secured to the waveguide 230 by welding the annular flange 506 to the first flattened portion 240a of the waveguide 230. The first flattened portion 240a provides for a large flat surface area for welding the first coupler 500 thereto in fluid-tight fashion. Various types of welding are contemplated, such as inertial friction welding, and other methods of fluid-tight attachment are also contemplated, such as brazing, soldering, diffusion bonding, etc.

With reference to FIGS. 4A and 4C, the second coupler 502 includes a main body 512, an annular flange 514 protruding radially outward from the main body 512, and an optional cylindrical extension 516 extending into the second transverse bore 236b. The second coupler 502 differs from the first coupler 500 by defining a linear channel 518 therethrough that fluidly connects the second transverse bore 236b and the return conduit 128b. As such, a distal end portion 231 of the return conduit 128b has an elbow bend whereas the distal end portion 229 of the inflow conduit 128a is linear. The second coupler 502 is secured to the distal body 234 of the waveguide 230 by welding the annular flange 514 thereof to the second flattened portion 140b of the waveguide 230. Further, the distal end portions 229, 231 of the inflow and return conduits 128a, 128b may be secured to the first and second couplers 500, 502, respectively, via any suitable connection, such as, for example, welding, gluing, soldering, compression fit, etc.

The couplers 500, 502 are sealed to the first and second transverse bores 236a, 236b of the waveguide 230 without leaks, providing a large enough flow rate to cool the blade 282 quickly, while keeping manufacturing costs down. It is contemplated that the couplers 500, 502 may be welded to the waveguide 230 at any suitable location of the waveguide 230, such as, for example, adjacent the proximal end, the distal end, or any suitable location therebetween. Although couplers 500, 502 are shown coupled to first and second transverse bores 236a, 236b, respectively, it is contemplated that this configuration be reversed, e.g., wherein couplers 502, 500 are shown coupled to first and second transverse bores 236a, 236b, respectively, or that the same type of coupler be utilized for both transverse bores 236a, 236b, e.g., two couplers 500 or two couplers 502.

Referring also to FIGS. 1 and 2, in use, upon activation of cooling, cooling system 120 is configured to pump fluid, using the one or more fluid pumps 122, from fluid reservoir 124, through inflow conduit 128a, distally through longitudinal lumen 242, from longitudinal lumen 242 to longitudinal lumen 244 at the distal end portion of blade 282, proximally through longitudinal lumen 244, through outflow conduit 128b, and back to fluid reservoir 124. In this manner, cooling fluid is circulated substantially entirely along the length of blade 282 to facilitate cooling blade 282. The cooling fluid may be saline, water, or other suitable fluid.

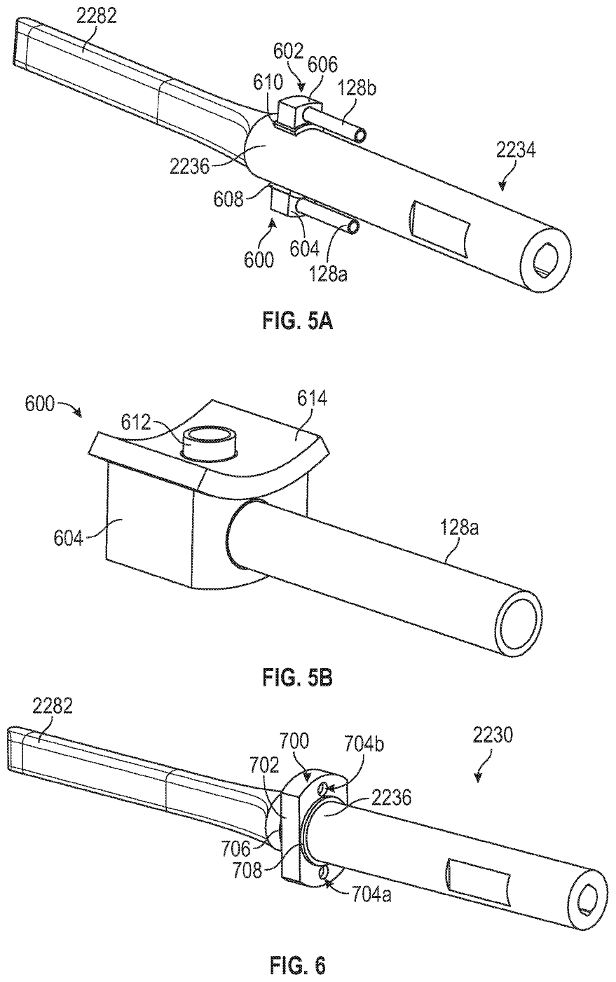

With reference to FIGS. 5A and 5B, another type of coupler 600, 602 for coupling inflow and outflow conduits 128a, 128b to a distal body 2234 including a blade 2282 of another waveguide 2230 is shown. Distal body 2234 is similar to and may include any of the features of distal body 234 (FIGS. 1 and 4A-4C) except as explicitly contradicted below, and couplers 600, 602 are similar to and may include any of the features of first coupler 500 (FIGS. 4A and 4B) except as explicitly contradicted below.

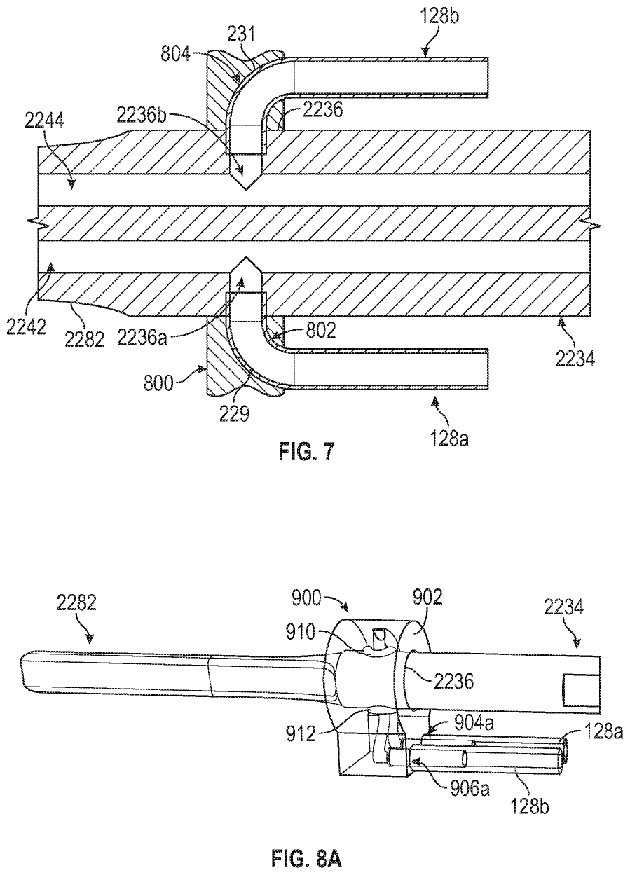

Distal body 2234, instead of having flattened portions, has an annular portion 2236 extending around the entire circumference thereof. Annular portion 2236 need not be distinct from the remainder of distal body 2234; that is, annular portion 2236 may be defined by the generally cylindrical configuration of distal body 2234. First and second couplers 600, 602 are secured to the distal body 2234 of the waveguide 2230 on opposite radial sides of the annular portion 2236. Each of the first and second couplers 600, 602 includes a respective main body 604, 606, a flange 608, 610 protruding outwardly from the main body 604, 606, and an optional cylindrical extension 612 (FIG. 5B, only the cylindrical extension of first coupler 600 is shown) extending into the transverse bores 2236a, 2236b (FIG. 7) defined in the distal body 2234.

The difference between the first and second couplers 600, 602 and the first coupler 500 of FIGS. 4A and 4B is that the flange 608, 610 of the first and second couplers 600, 602 has an arcuate shape such that an inner-facing surface 614 (FIG. 5B) thereof is concave and of substantially similar radius of curvature as annular portion 2236. The inner-facing surface 614 of the flanges 608, 610 thus sits flush with the annular portion 2236 whereby the flanges 608, 610 are welded to the annular portion 2236 in fluid-tight fashion. Various types of welding are contemplated, such as inertial friction welding, and other methods of fluid-tight attachment are also contemplated, such as laser welding, brazing, soldering, diffusion bonding, etc.

With reference to FIG. 6, another type of coupler 700 for coupling inflow and return conduits 128a, 128b (FIGS. 5A and 5B) to the blade 2282 of waveguide 2230 of FIG. 5A is shown. Coupler 700 is similar to and may include any of the features of couplers 600, 602 (FIGS. 5A and 5B) except as explicitly contradicted below. More specifically, coupler 700 provides a single, annular component that functions as the coupler for both lumens, rather than providing separate couples 600, 602 (FIGS. 5A and 5B).

Coupler 700 includes an annular body 702 that surrounds annular portion 2236 of waveguide 2230 and defines first and second L-shaped channels 704a, 704b therethrough configured for fluid communication with respective first and second lumens 2242, 2244 (FIG. 7) of waveguide 2230 and respective inflow and return conduits 128a, 128b (FIG. 7). Coupler 700 may be slid over waveguide 2230 from the proximal or distal end thereof into position to surround annular portion 2236 of waveguide 2230. Coupler 700 further includes a pair of annular flanges 706, 708 that protrude radially outwardly from opposite sides of the annular body 702. The annular flanges 706, 708 sit flush with the annular portion 2236 of the waveguide 2230 whereby the annular flanges 706, 708 are welded to the annular portion 2236 in fluid-tight fashion. Various types of welding are contemplated, such as laser welding, and other methods of fluid-tight attachment are also contemplated, such as brazing, soldering, diffusion bonding, etc. It is also contemplated that coupler 700 include flattened inner annular surfaces to enable welding to flattened portions of waveguide 2230, similarly as detailed above with respect to couplers 500, 502 (FIGS. 4A-4C).

With reference to FIG. 7, another type of coupler 800 for coupling inflow and outflow conduits 128a, 128b to the blade 2282 of waveguide 2230 of FIG. 5A is shown. With respect to coupler 800, the distal end portion 229, 231 of each of the inflow and return conduits 128a, 128b are directly connected to transverse bores 2236a, 2236b as shown. The coupler 800 is an elastomeric over-mold having an annular shape that surrounds the annular portion 2236 of the distal body 2234 of the waveguide 2230. The elastomeric over-mold 800 is over-molded to the distal end portion 229, 231 of the conduits 128a, 128b and the annular portion 2236 of the distal body 2234 of the waveguide 2230. The elastomeric over-mold 800 secures these components and acts as a seal to keep fluids and gasses from leaking out from between the bores 2236a, 2236a and the conduits 128a, 128b. The elastomeric over-mold 800 contains two curved lumens 802, 804 through which the conduits 128a, 128b are routed to the transverse bores 2236a, 2236b. The elastomeric over-mold 800 may further function as a sleeve seal for establishing a seal between waveguide 2230 and an inner sleeve of elongated assembly 200 (FIG. 1), thus inhibiting fluid ingress proximally through elongated assembly 200 (FIG. 1) and about waveguide 2230.

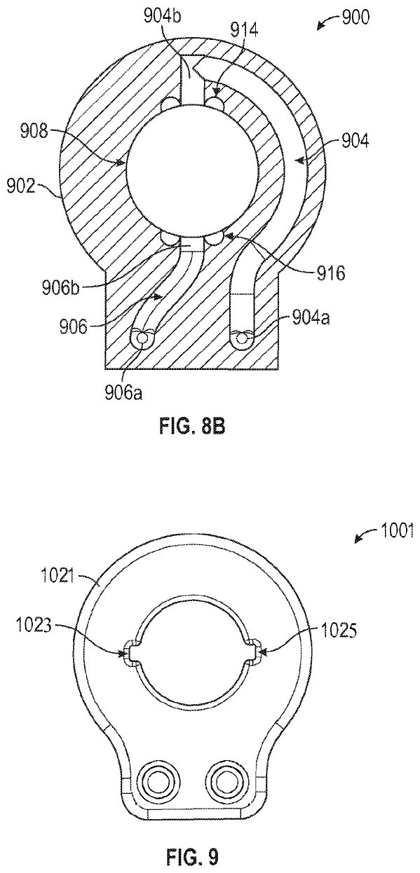

With reference to FIGS. 8A and 8B, another type of coupler 900 for coupling inflow and return conduits 128a, 128b to the blade 2282 of the distal body 2234 of the waveguide 2230 is shown. The coupler 900 includes a manifold 902 that surrounds the annular portion 2236 of the waveguide 2230 and which defines first and second separate channels 904, 906 therethrough. The manifold 902 may be printed (or otherwise formed) as a single part or formed from two halves that are sealed together by appropriate means (welding, glue, fasteners, etc.). The manifold 902 defines a central passage 908 therethrough. The manifold may be fixed to the annular portion 2236 of the waveguide 2230 via frictional engagement or other suitable fastening means, e.g., welding.

The first channel 904 has a first opening 904a configured for entry of the inflow conduit 128a into the manifold 902, and a second opening 904b in fluid communication with the central passage 908 of the manifold 902. The second channel 906 has a first opening 906a configured for entry of the return conduit 128b into the manifold 902, and a second opening 906b in fluid communication with the central passage 908 of the manifold 902. The first channel 904 extends from the first opening 904a thereof up and partially around the manifold 902 to the second opening 904b. As such, the second openings 904b, 906b are diametrically opposed to one another and the first opening 904a, 906a are adjacent and parallel with one another.

The coupler 900 further includes a pair of seals, such as, for example, O-rings 910, 912 that are seated in corresponding annular recesses 914, 916 formed in the manifold 902 and positioned around the second openings 904b, 906b of the respective first and second channels 904, 906. The O-rings 910, 912 seal the manifold 902 to the waveguide 2230. It is contemplated that additional seals (not explicitly shown) may be over-molded onto the blade 2282 or the manifold 902.

With reference to FIG. 9, another type of coupler 1000 for coupling inflow and return conduits 128a, 128b to the blade 2282 of waveguide 2230 (FIG. 8A) is shown. The coupler 1000 is similar to and may include any of the features of coupler 900 (FIGS. 8A and 8B) except as explicitly contradicted below.

Coupler 1000 includes a manifold 1002, similar to manifold 902, but for a pair of locating slots 1004, 1006 formed therein. The locating slots 1004, 1006 are configured for receipt of corresponding surface protrusions (not explicitly shown) extending from opposite sides of the waveguide 2230. The locating slots 1004, 1006 orient the coupler 1000 during assembly to the waveguide 2230.

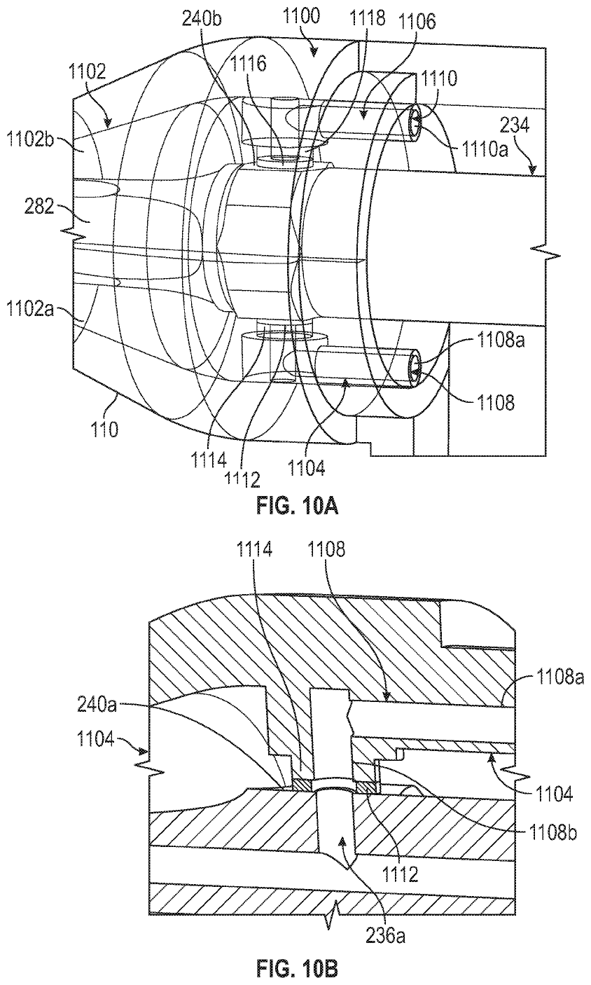

With reference to FIGS. 10A and 10B, another type of coupler 1100 for coupling inflow and return conduits 128a, 128b to the blade 282 of waveguide 230 (FIGS. 4A-4C) is shown. The coupler 1100 includes a housing 1102 that surrounds the waveguide 230 while providing a gap 1104 (FIG. 10B) therebetween. The housing 1102 may be fabricated from two housing halves 1102a, 1102b that are held together and fixed in place by any suitable fastening mechanism, such as, for example, fasteners, glue, ultrasonic welding, etc. The first and second housing halves 1102a, 1102b have respective first and second flanges 1104, 1106 each defining an L-shaped channel 1108, 1110 therethrough. As an alternative to a multi-piece housing 1102, the housing 1102 may be a single piece that is slid proximally over the distal end of waveguide 230 (FIGS. 4A-4C) into position.

The L-shaped channel 1108 of the first flange 1104 has a first opening 1108a configured for entry of the inflow conduit 128a (FIG. 4A) into the first flange 1104, and a second opening 1108b (FIG. 10B) aligned with the first transverse bore 236a. A seal, such as, for example, a gasket 1112 is positioned between an end portion 1114 of the first flange 1104 and the first flattened portion 240a of the waveguide 230. Similarly, the L-shaped channel 1110 of the second flange 1106 has a first opening 1110a configured for entry of the return conduit 128b (FIG. 4A) into the second flange 1106, and a second opening (not numerated) aligned with the second transverse bore 236b (FIG. 4A). A seal, such as, for example, another gasket 1116 is positioned between an end portion 1118 of the second flange 1106 and the second flattened portion 240b of the waveguide 230. The gaskets 1112, 1116 are compressed and held in position by the coupling between the housing halves 1102a, 1102b. The housing 110 (FIGS. 1 and 10A) may also facilitate holding the housing halves 1102a, 1102b together, and therefore compressing the gaskets 1112, 1116.

While several aspects and features of the disclosure are detailed above and shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular configurations. Those skilled in the art will envision other modifications within the scope and spirit of the claims appended hereto.

1. An ultrasonic surgical system, comprising:
an ultrasonic waveguide having a distal body defining:
first and second longitudinal lumens in fluid communication with one another; and
first and second transverse bores in fluid communication with the respective first and second longitudinal lumens;
an inflow conduit and an outflow conduit that are fluidly coupled with the respective first and second transverse bores to enable an inflow of fluid into the first longitudinal lumen and an outflow of fluid from the second longitudinal lumen; and
at least one coupler configured to couple the inflow and outflow conduits to the distal body and form a fluid-tight seal between the inflow and outflow conduits and the respective first and second transverse bores.

2. The ultrasonic surgical system according to claim 1, wherein the at least one coupler is welded to the distal body.

3. The ultrasonic surgical system according to claim 2, wherein the at least one coupler includes:
a first main body defining a first channel therethrough that fluidly couples the first transverse bore and the inflow conduit to one another; and
a first flange protruding radially outward from the first main body and welded to the distal body.

4. The ultrasonic surgical system according to claim 3, wherein the distal body has an annular portion, and the first flange is curved such that the first flange is flush with the annular portion.

5. The ultrasonic surgical system according to claim 3, wherein the distal body has a flattened portion, and the first flange is planar such that the first flange is flush with the flattened portion.

6. The ultrasonic surgical system according to claim 3, wherein the at least one coupler includes:
a first coupler having the first main body and the first flange; and
a second coupler having:
a second main body defining a second channel therethrough that fluidly couples the second transverse bore and the outflow conduit to one another; and
a second flange protruding radially outward from the second main body and welded to the distal body.

7. The ultrasonic surgical system according to claim 6, wherein the first channel is linear or L-shaped and has a distal end portion of the inflow conduit received therein, and the second channel is the other of linear or L-shaped and has a distal end portion of the outflow conduit received therein.

8. The ultrasonic surgical system according to claim 3, wherein the at least one coupler further includes a cylindrical extension extending from the first main body and received within the first transverse bore.

9. The ultrasonic surgical system according to claim 3, wherein the first main body is a ring that extends around a circumference of the distal body, the first main body defining a second channel therethrough that fluidly couples the second transverse bore and the outflow conduit to one another.

10. The ultrasonic surgical system according to claim 9, wherein the at least one coupler includes a second flange protruding radially outward from the first main body and welded to the distal body, the first and second flanges extending from the first main body in opposite directions from one another.

11. The ultrasonic surgical system according to claim 1, wherein the ultrasonic waveguide includes a proximal body configured to connect to an ultrasonic transducer, wherein the distal body has a blade through which the first and second longitudinal lumens extend.

12. The ultrasonic surgical system according to claim 11, further comprising:
a cooling system configured to pump cooling fluid through the inflow conduit into the first longitudinal lumen and/or pump cooling fluid through the second longitudinal lumen into the outflow conduit;
a housing at least partially supporting the cooling system; and
an elongated assembly extending distally from the housing, the elongated assembly including the ultrasonic waveguide.

13. The ultrasonic surgical system according to claim 1, wherein the at least one coupler includes an elastomeric over-mold overmolded to a distal end portion of each of the inflow and outflow conduits and the distal body.

14. The ultrasonic surgical system according to claim 1, wherein the at least one coupler includes:
a manifold that surrounds the distal body and defines first and second channels; and
a pair of compressible seals that form a fluid-tight seal between the first channel and the first transverse bore and the second channel and the second transverse bore.

15. The ultrasonic surgical system according to claim 14, wherein the manifold defines a pair of annular recesses positioned around a respective opening of the first and second channels, the pair of seals being O-rings seated in the corresponding annular recesses.

16. The ultrasonic surgical system according to claim 1, wherein the at least one coupler includes:
a first housing half surrounding a first section of the distal body and having a first flange defining a channel, the channel of the first flange configured to fluidly couple the first transverse bore and the inflow conduit to one another; and
a second housing half surrounding a second section of the distal body and having a second flange defining a channel, the channel of the second flange configured to fluidly couple the second transverse bore and the outflow conduit to one another.

17. The ultrasonic surgical system according to claim 16, wherein the at least one coupler further includes:
a first gasket compressed between an end portion of the first flange and an outer surface of the first section of the distal body to form a fluid-tight seal between the channel of the first flange and the first transverse bore; and
a second gasket compressed between an end portion of the second flange and an outer surface of the second section of the distal body to form a fluid-tight seal between the channel of the second flange and the second transverse bore.
